(19) United States
(12) Patent Application Publication
(10) Pub. No.: US 2012/0028899 A1
(43) Pub. Date: Feb. 2, 2012

(54) COMPOSITION AND PROCESS FOR SYNTHESIZING TENSE AND RELAXED STATE POLYMERIZED HEMOGLOBIN

(76) Inventor: Andre Francis Palmer, Bexley, OH (US)

(21) Appl. No.: 12/847,868

(22) Filed: Jul. 30, 2010

(51) Int. Cl. A61K 38/42; C12N 5/07; A61P 9/00; C07K 14/805

(52) U.S. Cl. 514/13.5; 530/385; 435/375

(57) ABSTRACT

Described herein is a composition and process for synthesizing a hemoglobin-based oxygen carrier (HBOC) composition that includes a polymerized hemoglobin locked in at least one of the tense or relaxed quaternary state and has a molecular weight of at least about 500 kDa. The PolyHb may have a cross-linker to hemoglobin molar ratio of at least about 20:1. The polymerized hemoglobin is useful for delivering oxygen to cells, such as for treatment of anemic conditions in a subject, perfusion of tissues or organs, and supplementing cell culture media in cell culturing systems.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. R01HL078840 and R01DK070862 awarded by the National Institutes of Health.

### FIELD

The present invention relates generally to hemoglobin-based oxygen carriers (HBOCs) and more particularly to polymerized hemoglobin (PolyHb) molecules.

### BACKGROUND

Mammalian cells require a regular and controlled supply of O₂ to meet their metabolic needs. In the body, O₂ is normally delivered to cells by red blood cells (RBCs) circulating in the cardiovascular system. However, in some instances, such as severe anemia caused by injury or disease, the ability of the cardiovascular system to deliver O₂ via RBCs is compromised. In cell culture systems, O₂ delivery is generally limited by the solubility of O₂ in aqueous cell culture media. Therefore, compositions capable of the regular and controlled delivery of O₂ to cells, tissues and organs in an animal or human subject or in a cell culturing system are needed.

Some HBOCs are being developed as RBC substitutes to deliver O₂ to cells. With regard to treating subjects, vasoconstriction and hypertension are important side-effects in subjects transfused with previously developed HBOCs. Although the exact mechanism of vasoconstriction upon HBOC transfusion is not known, there are two major hypotheses in the literature, namely nitric oxide (NO) scavenging and oxygen oversupply.

The NO scavenging hypothesis is based on the ability of cell free hemoglobin (Hb) to either extravasate through the wall of blood vessels or to diffuse in close proximity to the blood vessel wall. Once inside or near the blood vessel wall, these molecules readily scavenge NO generated by the surrounding endothelial cell layer. In response, the surrounding smooth muscle layer constricts leading to vasoconstriction and an increase in the systemic blood pressure.

In the O₂ oversupply hypothesis, cell free Hb is thought to facilitate the diffusive transport of O₂ to the blood vessel wall. If too much O₂ is delivered to the surrounding tissues (overoxygenation), an autoregulatory response occurs in which the blood vessel constricts (vasoconstriction) decreasing the available surface area for O₂ transport. This response increases vascular resistance leading to the development of systemic hypertension. Therefore, a HBOC composition that is capable of delivering O₂ to cells, tissues and organs in an animal or human subject that does have these side effects is needed.

As mentioned above, another current problem is the delivery of O₂ to cells grown in cell culture systems. It is well known that O₂ is sparingly soluble in aqueous media (˜0.2 mmol/L at 1 atm air, 37° C.). As a result, cell culture media in most cell culture systems need to be oxygenated to supraphysiological levels (>160 mm Hg) in order to deliver enough O₂ to cultured cells, which results in a portion of the cultured cells being exposed to hyperoxic conditions. Prolonged exposure to hyperoxic conditions will induce the formation of reactive O₂ species (ROS) which will eventually kill cells.

Many methods have been proposed and utilized to improve O₂ delivery in large scale cell culture systems, including re-design of cell culture bioreactors by computational modeling in order to alleviate mass transfer limitations, and supplementation of O₂ carriers (HBOCs and perfluorocarbon-based) into the cell culture media in order to increase the solubility of O₂ in aqueous media. Computational fluid dynamics has long been used to design and to optimize bioreactors by simultaneously modeling momentum and mass transfer. The use of O₂ carriers in the cell culture system, on the other hand, provides a biomimetic approach to recapitulate the in vivo oxygenation environment for many cell culture and tissue engineering applications.

In particular, hepatic hollow fiber (HF) bioreactors which constitute one type of bioartificial liver assist device (BLAD) suffer from O₂ limited transport mainly due to the low solubility of O₂ in the cell culture media, long diffusion pathlengths, and high demand for O₂ by the hepatocytes cultured in the extracapillary space (ECS). These devices are expected to bridge patients suffering from acute liver failure (ALF) towards native liver regeneration or orthotopic liver transplantation by providing sufficient global liver functions.

In vivo, blood enters the periportal region of liver sinusoid via the hepatic artery and portal vein at a mean pO₂ of ˜65 mm Hg and then leaves the sinusoid in the perivenous region via the central vein at a pO₂ of ˜25-35 mm Hg, forming an O₂ gradient along the length of the liver sinusoid. The O₂ gradient along the length of the liver sinusoid is one of the factors controlling the liver's varied metabolic and synthetic functions. For instance, hepatocytes exhibiting gluconeogenesis functionality seem to be localized in the periportal region (where the local pO₂ is highest in the sinusoid), while cytochrome P₄₅₀ activity is predominantly found in the perivenous region (where the local pO₂ is lowest in the sinusoid). The regulation of liver zonation is controlled at the transcriptional level, and it seems to be tightly coupled to the O₂ gradient via O₂-sensitive transcription factors. Therefore, provision of appropriate oxygenation to hepatocytes cultured within a BLAD at similar levels, including reproducing the O₂ gradient observed physiologically, should result in a better functioning device. Thus, a HBOC composition that is capable of reproducing physiological O₂ gradients and delivering O₂ to cells in cell culturing systems is needed.

### SUMMARY

In order to improve delivery of O₂ to cells and to limit the detrimental side effects of free Hb, the size (i.e., molecular weight [MW]) of the HBOC should be increased in order to lower the rate of acellular Hb extravasation, transport to the blood vessel wall, and subsequent NO scavenging; as well as, lower the extent of facilitated O₂ diffusion. Thus, described herein are high MW (at least about 500 kDa) PolyHb compositions that are synthesized in either the tense (T) or relaxed (R) quaternary states. The PolyHb compositions may have a cross-linker to Hb molar ratio of at least 20:1. Methods of using the PolyHb compositions to deliver oxygen to cells, such as in the treatment of anemia in a subject, perfusion of a natural or artificial tissue or organ, or the supplementation of cell culture media used in cell culturing techniques are also described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph demonstrating the pO₂ at various stages of the bHb polymerization process for 50:1 T- and 40:1 R-state PolybHb solutions.

FIG. 2 is an image of an SDS-PAGE of native bHb, 50:1 T- and 40:1 R-state PolybHb solutions.

FIG. 3 is a graph demonstrating the absolute MW distribution of native bHb, 50:1 T- and 40:1 R-state PolybHb solutions.

FIG. 4A is a graphical representation of a hepatic HF bioreactor system.

FIG. 4B is an expanded view of the HF bioreactor cartridge of FIG. 3A.

FIG. 4C is a graphical representation of the geometry of a single HF of FIG. 3B used in the computer simulations.

FIG. 5A is a graph illustrating the O₂ consumption rate of bHb and T-state PolybHb solutions normalized by the control (no HBOC supplementation).

FIG. 5B is a graph illustrating the O₂ consumption rate of bHb and R-state PolybHb solutions normalized by the control (no HBOC supplementation).

FIG. 6 is a graph illustrating the O₂ consumption rate of hHb/PolyhHb solutions normalized by the control (no HBOC supplementation).

FIG. 7A is a graph illustrating pO₂ profiles within a single HF (lumen, membrane and ECS) with varying heme concentration of bHb/PolybHb (100% [Heme]=15 g/dL of bHb).

FIG. 7B is a graph illustrating ECS zonation with a 100% heme concentration.

FIG. 8 is a graph illustrating pO₂ profiles within a single HF (lumen, membrane and ECS) for hHb/PolyhHb at varying heme concentrations where each rectangle represents the region depicted in FIG. 3C.

FIG. 9 is a graph illustrating ECS zonation within the HF bioreactor for hHb/PolyhHb with a 100% heme concentration.

### DETAILED DESCRIPTION

One embodiment of the invention is directed to high MW PolyHb compositions locked in a desired quaternary state, i.e., tensed or relaxed state (T-state PolyHb or R-state PolyHb), which can function as a HBOC. A majority of the PolyHb in the composition has a high MW of at least about 500 kDa and in another embodiment the high MW is at least about 1,000 kDa or at least about 10,000 kDa. The high MW PolyHb compositions may have an upper limit of about 50,000 kDa. In one embodiment, more than 50% of the PolyHb has a high MW and in other embodiments, at least about 75% of the PolyHb has a high MW, or at least about 85% of the PolyHb has a high MW, or at least about 95% of the PolyHb has a high MW, or at least about 100% of the PolyHb has a high MW.

In addition, the PolyHb may have a cross-linker to Hb tetramer molar ratio, referred to herein as the cross-link density, of at least about 20:1 or at least about 30:1, or at least about 40:1 or at least about 50:1 or at least about 60:1 and may optionally range between any of these cross-linking densities. For example, the cross-linking density may be in a range from about 20:1 to about 60:1. In one embodiment, cross-linking density is at least about 40:1 and may be in a range from about 40:1 to about 60:1 and in another embodiment, the cross-linking density is at least about 50:1 and may be in a range from about 50:1 to about 60:1. The MW and/or cross-link density of the PolyHb compositions affect their biophysical characteristics, which directly determine viscosity, colloid osmotic pressure, and oxygen transport properties and indirectly affect hypertension and vasoconstriction when infused. As shown in the examples below, higher MW density PolyHb compositions having higher cross-link densities generally have improved biophysical characteristics relative to PolyHb compositions having a lower MW and/or lower cross-linking density.

Another aspect of the invention is directed to a process for synthesizing the PolyHb compositions described above, i.e. a PolyHb locked in the desired quaternary state (i.e., tensed or relaxed state), having a MW of at least about 500 kDa. The PolyHb may have a cross-linking density of at least about 20:1. The process includes adjusting the pO₂ of a solution containing Hb to a desired level, polymerizing the Hb with a cross-linker having a desired cross-linking density while maintaining the desired pO₂, and purifying the PolyHb having the desired molecular weight.

In one embodiment, the Hb containing solution includes Hb isolated from RBCs, such as by using tangential flow filtration (TFF). For example, whole blood is collected. Collected blood may also be purchased from suitable vendor such as Quad Five (Ryegate, Mo.). The RBCs of the collected whole blood are lysed. For example, RBCs are initially washed with a suitable buffer such as isotonic saline solution. The washed RBCs are subsequently lysed with a suitable hypotonic buffer. The lysate is filtered through a glass chromatography column packed with glass wool to remove the majority of cell debris. The clarified RBC lysate is then passed through a TFF hollow fiber (HF) cartridge to remove additional cell debris and impurity proteins. The purified Hb is collected and concentrated to yield the precursor Hb for PolyHb synthesis. The source of the RBC's from which Hb is purified, i.e., vertebrate, invertebrate, etc., will depend on the desired end use of the PolyHb. It is further contemplated that other sources of purified Hb may used in the PolyHb synthesis described below, such as recombinant Hb or purified Hb supplied by a vendor.

Isolated Hb is used to synthesize PolyHb that is locked in a desired quaternary state, (T-state or R-state, respectively), or combinations thereof. T-state and R-state PolyHbs are synthesized as follows: First, a Hb containing solution is made by diluting isolated Hb with a suitable buffer such as 20 mM phosphate buffer (PB) (pH 8.0) to a suitable concentration such as 0.1-1 mM. Next the pO₂ of the Hb containing solution is adjusted to the level necessary to obtain the desired T- or R-state PolyHb. Then the Hb is polymerized with a cross-linking agent while maintaining the desired pO₂ level. The polymerization reaction is quenched with a strong reducing agent to reduce any free aldehyde groups and to reduce any free Schiff bases in solution. PolyHb having the desired MW is then collected by purification from unwanted elements, such as Hb outside of the desired MW range.

For the synthesis of T-state PolyHb, the pO₂ of the Hb containing solution is decreased to the desired level such that between 0% and 10% of the Hb is saturated with O₂. Preferably, 0% of the Hb is saturated so as to yield a PolyHb composition in which 100% of the Hb is locked in the T-state. For example, the pO₂ level may be decreased to the desired level using any gas exchange technique, e.g. purging the solution with an inert gas, adding an oxygen scavenging agent to the solution, or using a gas-liquid exchanger and combinations thereof. For instance, the Hb solution may be subjected to alternative vacuum and argon purging cycles under continuous stirring for 4 hours to remove the majority of O₂. The gas exchange step may be repeated as needed for a prolonged period of time in order to lower the pO₂ of the Hb solution. While the gas exchange step may remove a majority of the O₂ from the Hb containing solution, lowering the pO₂ to 0 mm Hg to so that 0% of the Hb is saturated with O₂ also requires the addition of an oxygen scavenging agent such as Na₂S₂O₄ to remove all remaining residual O₂. For example, a 1.5 mg/mL stock solution of the oxygen scavenging agent can be infused into the Hb containing solution in doses as needed under constant stirring, while the pO₂ of the Hb containing solution is monitored such as after the infusion of each dose using a blood gas analyzer like the RapidLab 248 Blood Gas Analyzer (Siemens USA, Malvern, Pa.). Once the pO₂ is at the desired level (e.g., around about 0 mm Hg), the infusion of the O₂ scavenging agent is terminated. Next, a deoxygenated cross-linker, such as glutaraldehyde (70%), is added to the deoxygenated Hb containing solution to polymerize the Hb at cross-linker to Hb tetramer molar ratios, also referred to herein as the cross-linking density, ranging from 20:1 to 60:1. The pO₂ is maintained at the desired level during the polymerization step. The polymerization reaction is allowed to continue for 2-24 hours in the dark at 37° C. and then quenched with a strong reducing agent such as 20 mL of 2 M NaBH₄ in PB buffer (20 mM, pH 8.0) to yield T-state PolyHb. This process yields a T-state PolyHb composition in which between 0% and about 10% of the Hb in the T-state PolyHb is saturated with O₂, i.e., about 90% to about 100% of the Hb in the PolyHb is locked in the T-state. Preferably, about 100% of the Hb in the PolyHb is locked in the T-state.

R-state PolyHb is synthesized in a similar fashion except that the Hb containing solution is purged with pure O₂ until the desired pO₂ level is achieved. For example, in one embodiment, the Hb containing solution is purged with pure O₂ for 2 hours in an ice bath, while the pO₂ is continuously monitored with a blood gas analyzer. Once the pO₂ is at the desired level such that the Hb is about 90% to 100% saturated with O₂, a cross-linker such as glutaraldehyde is added to the solution while maintaining the pO₂ in the desired range. The cross-linker is added to the solution at cross-linker to Hb tetramer molar ratios, also referred to herein as the cross-linking density, ranging from 20:1 to 60:1. At the end of the polymerization period 2-24 hours in the dark at 37° C., a reducing agent, such as 5 mL of 8 M NaCNBH₃ in PB buffer (20 mM, pH 8.0), is added to the reaction mixture to reduce the resultant Schiff bases and methemoglobin (metHb). The reaction mixture is then stirred on ice for another 30 minutes, and the reaction is quenched with a strong reducing agent such as with 20 mL of 2M NaBH₄ in PB buffer (20 mM, pH 8.0) to yield R-state PolyHb. This process yields a R-state PolyHb composition in which a majority of the Hb in the PolyHb is locked in the R-state. Preferably, between about 90% and 100% of the Hb in the PolyHb is saturated with O₂, i.e., are locked in the R-state. Preferably, about 100% of the Hb in the PolyHb is locked in the R-state.

It is also possible to make PolyHb solutions with varying T- and R-state character. This can be done by equilibrating the Hb solution using any gas-liquid exchange method with an inert gas/O₂ mixture that can vary anywhere between 0% to 100% O₂. The Hb solution can then be polymerized and quenched as previously described. For example, a PolyHb solution made with 21% O₂ may be acceptable. It is also possible to make PolyHb mixtures composed of combinations of 100% T-state PolyHb and 100% R-state PolyHb.

Cross-linking agents in addition to glutaraldehyde include succindialdehyde, activated forms of polyoxyethylene and dextran, α-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bisimidate class, the acyl diazide class or the aryl dihalide class, and combinations thereof.

The PolyHb has a MW of least about 500 kDa and an upper limit of about 50,000 kDa. Thus, the process includes the step of collecting PolyHb having the desired MW range. The collecting step may include separating or purifying PolyHb having the desired MW range or making the PolyHb free from undesirable elements such as Hb having a MW outside of the desired range. For example, the PolyHb solution may be clarified such as by being passed through a glass chromatography column packed with glass wool to remove large particles. The clarified PolyHb solution is then separated into distinct molecular mass fractions using known separation methods such as passing the clarified PolyHb solution through a TFF HF cartridge selected to collect PolyHb having the desired MW, such as a 500 kDa cartridge (Spectrum Labs). The retentate contains PolyHb molecules that are larger than the desired MW, whereas the filtrate mostly contains PolyHb molecules that are smaller than the desired MW, e.g. 500 kDa. The PolyHb solution may then be subjected to as many cycles of dialfiltration with an appropriate buffer as needed in order to remove all impurities less than the desired MW. The MW of the PolyHb can be controlled by passing the clarified PolyHb solution through TFF HF cartridges having different pore sizes. After separation of the desired fraction, the filtrate may subsequently be concentrated such as with a 100 kDa TFF HF cartridge (Spectrum Labs). The MW distribution of the PolyHb may be confirmed by known methods such as SDS-PAGE analysis or size exclusion chromatography coupled with multi-angle static light scattering.

After polymerization, PolyHb is suspended in a buffer along with the reduced cross-linker and excess quenching agent. Some cross-linkers like glutaraldehyde and some quenching agents (i.e., NaBH₄ and NaCNBH₃) are cytotoxic; therefore, the PolyHb solution may be buffer exchanged with another buffer such as modified lactated Ringer solution [115 mM NaCl (USP), 4 mM KCl (USP), 1.4 mM CaCl₂ H₂O (USP), 13 mM NaOH (NF), 27 mM sodium lactate (USP), and 2 g/L N-acetyl-L-cysteine (USP)].

In use, PolyHb is utilized as a HBOC for delivery of oxygen to cells without the substantial side effects that result from other HBOCs, such as vasoconstriction, hypertension, and decreased tissue oxygenation. PolyHb may be used as a blood substitute in a subject that has a need for increased capacity to deliver oxygen to tissues such as a patient suffering from anemia that results from an injury or disease. For this use, PolyHb is infused into the circulatory system of the subject, such as through intravenous or intraarterial infusion through a catheter. PolyHb may also be mixed with blood compositions, with includes whole blood, plasma, or blood fractions, as well as, crystalloid solutions and other plasma expanders prior to infusion into the subject. In this regard, exemplary conditions in which PolyHb may be useful include: treatments for wounds, anemia, head injury, hemorrhage, hypovolemia, ischemia, cachexia, sickle cell crisis and stroke; enhancing cancer treatments; enhancing cell/organ/tissue preservation; stimulating hematopoiesis; improving repair of physically damaged tissues; alleviating cardiogenic shock; shock resuscitation; and cosmetics. In addition to infusion into the general circulatory system of a subject, PolyHb may also be used to perfuse an individual organ or tissue, such as to maintain an organ or tissue during transplantation or other procedure that restricts regular blood flow to the organ or tissue.

Another use for the PolyHb is in cell culture. For this use, growth media is supplemented with PolyHb. For example, the PolyHb may be included in the circulating growth medium of a hepatic HF bioreactor to improve oxygen delivery to cells cultured therein. The PolyHb then delivers O₂ to cultured cells within the bioreactor to thus decrease hypoxic zones.

Various aspects of the present invention are further illustrated by the following non-limiting examples.

### Example 1

The following is a description of the specific methods used to produce compositions in which a majority of the ultrahigh MW bovine PolyHb (PolybHb) has a MW of at least about 500 kDa and about 100% of the Hb is locked in at least the T- or R-states. The PolybHb compositions produced by these methods were then tested in later examples.

Materials used in example 1—Glutaraldehyde (70%), NaCl, KCl, NaOH, Na₂S₂O₄, NaCl, KCl, CaCl₂-2H₂O, NaOH, sodium lactate, N-acetyl-L-cysteine, NaCNBH₃ and NaBH₄ were purchased from Sigma-Aldrich (Atlanta, Ga.). Sephadex G-25 resin was purchased from GE Healthcare (Piscataway, N.J.). KCN, K₃Fe(CN)₆, and all other chemicals were purchased from Fisher Scientific (Pittsburgh, Pa.).

In preparation for experiments, all glassware and plasticware were immersed in 1 mol/L NaOH solution for more than 6 hours to degrade any endotoxin present, followed by thorough rinsing with HPLC grade water.

Hb purification—Fresh bovine blood stored in 3.8% sodium citrate solution at a final concentration of 90:10 v/v (blood:sodium citrate solution) was purchased from Quad Five (Ryegate, Mo.). Bovine Hb (bHb) was purified via TFF from bovine RBC lysate. Bovine RBCs were initially washed 3 times with 3 volumes of isotonic saline solution (0.9%) at 4° C. Bovine RBCs were subsequently lysed on ice with 2 volumes of hypotonic, 3.75 mM phosphate buffer (PB) at pH 7.4 for 1 h. The RBC lysate was then filtered through a glass column packed with glass wool to remove the majority of cell debris. Clarified RBC lysate was then passed through 50 nm and 500 kDa TFF HF cartridges (Spectrum Labs, Rancho Dominguez, Calif.) to remove additional cell debris and impurity proteins. Purified Hb was collected and concentrated on a 100 kDa TFF HF cartridge (Spectrum Labs) to yield the raw material for synthesis of PolybHb.

Polymerization of bHb—To generate fully deoxygenated or T-state Hb, 30 g of purified Hb was diluted with phosphate buffer (PB) (20 mM, pH 8.0) to yield 1200 mL of Hb solution. The Hb solution was placed inside an airtight bottle and connected to a vacuum manifold. The entire system was kept below 4° C. in an ice water bath. The Hb solution was then subjected to several cycles of vacuum and argon (Ar) purging to remove the majority of O₂ from solution. After 4 h of vacuum and Ar cycling, Na₂S₂O₄ solution (1.5 mg/mL) was titrated into the Hb solution with a syringe pump (Razel Scientific, St. Albans, Vt.), while the pO₂ of the solution was simultaneously measured using a RapidLab 248 (Siemens, Malvern, Pa.) blood gas analyzer until the pO₂ of the Hb solution attained a value of 0 mm Hg. At this point, an additional 30 mL of 1.5 mg/mL Na₂S₂O₄ solution was added to the T-state Hb solution to maintain the pO₂ at 0 mm Hg during and after the polymerization reaction. A 30 mL syringe was used to titrate glutaraldehyde preequilibrated with Ar into the sealed glass bottle under continuous stirring. In this example, the T-state polymerization reaction was conducted at a 50:1 molar ratio of glutaraldehyde to Hb.

Relaxed (R) state Hb was prepared in a similar manner to T-state Hb using the same vacuum manifold system. 1500 mL of 0.3 mmol/L Hb solution was saturated with pure O₂ for 2 h in an ice-water bath and the pO₂ was monitored using a RapidLab 248 blood gas analyzer. When the pO₂ measured was well over the 749 mm Hg measurement range of the RapidLab 248 blood gas analyzer, a 30 mL syringe was used to titrate glutaraldehyde in the sealed glass bottle under continuous stirring. In this example, the R-state polymerization reaction was conducted at a 40:1 molar ratio of glutaraldehyde to Hb.

The resulting T- or R-state Hb solutions were then allowed to react with glutaraldehyde in the dark at 37° C. for 2 h, and were stirred and equilibrated with either pure Ar (T-state PolyHb) or O₂ (R-state PolyHb). At the end of the 2 hour reaction period, for the T-state PolyHb solution, 20 mL of 2 M NaBH₄ in PB buffer (20 mM, pH 8.0) was injected into the glass bottle to quench the polymerization reaction. For the R-state PolyHb solution, 5 mL of 8 M NaCNBH₃ in PB buffer (20 mM, pH 8.0) was injected into the glass bottle to reduce the Schiff base and reduce the metHb level of the PolyHb solution. The PolyHb solution was continuously stirred for 30 min in an ice-water bath. Subsequently, 20 mL of 2 M NaBH₄ in PB buffer (20 mM, pH 8.0) was injected into the glass bottle to quench the polymerization reaction. The pO₂ of the Hb solution before polymerization, after polymerization, and after quenching with NaBH₄ was measured using a RapidLab 248 blood gas analyzer. All reactions were repeated in triplicate.

Clarification and separation of PolyHb solutions—Initially, each PolyHb solution was clarified by filtering it through a glass column packed with glass wool that had been autoclaved at 250° C. for 30 min in order to degrade any endotoxin present in the glass wool. The clarified PolyHb solution was then separated into two distinct MW fractions with a 500 kDa TFF HF cartridge (Spectrum Labs). The retentate mostly contained PolyHb molecules that were larger than 500 kDa, while the filtrate contained PolyHb molecules less than 500 kDa.

Buffer exchange of PolyHb solutions—After clarification and separation, the PolyHb was suspended in PB buffer along with reduced glutaraldehyde and excess NaBH₄ (and excess NaCNBH₃ for R-state PolyHb). The PolyHb solution underwent buffer exchange to remove cytotoxic glutaraldehyde, NaCNBH₃ and NaBH₄ with a modified lactated Ringer's solution (NaCl 115 mmol/L, KCl 4 mmol/L, CaCl₂-2H₂O 1.4 mmol/L, NaOH 13 mmol/L, sodium lactate 27 mmol/L and N-acetyl-L-cysteine 2 g/L). The buffer exchange was conducted using an AKTA Explorer 100 system controlled by Unicorn 5.1 software (GE Healthcare). An XK 50/30 (300 mm in length, 50 mm I.D.) column (GE Healthcare) was packed with 500 mL of Sephadex G-25 medium resin at room temperature. After equilibrating the column with modified lactated Ringer's solution at a flow rate of 8 mL/min, the PolyHb solution was injected into the XK 50/30 column via a superloop (50 mL, GE Healthcare) at a flow rate of 5 mL/min. A hundred milliliters of sample was injected each time and then eluted with modified lactated Ringer's solution. The protein concentration was detected at a wavelength of 280 nm, while the salt concentration was monitored with a conductivity detector. During the buffer exchange process, the UV signal increased as PolyHb eluted from the column, while the conductivity decreased when reduced glutaraldehyde and NaBH₄ (and NaCNBH₃ for R-state PolyHb) eluted from the column. The buffer exchanged PolyHb solution was collected as the LTV signal increased, but before the conductivity signal decreased. The PolyHb fraction was then concentrated with a 100 kDa TFF HF cartridge (Spectrum Labs).

MetHb level and protein concentration of PolyHb solutions—The metHb level of PolyHb solutions was measured via the cyanomethemoglobin method. Total protein concentration was measured using the Bradford method using the Coomassie Plus protein assay kit (Pierce Biotechnology, Rockford, Ill.).

SDS-PAGE of PolyHb solutions—The MW distribution of PolyHb solutions was initially assessed via gel electrophoresis using a Mini-PROTEAN 3 Cell (Bio-Rad; Hercules, Calif.). All samples were mixed with an equal volume of sample buffer (Bio-Rad) containing 5% v/v mercaptoethanol, and then boiled for 5 min. A 4% stacking gel with a 12% resolving gel was assembled on a minivertical gel apparatus and each lane was loaded with 25 mg of protein. The gel was run at 120 V for approximately 1 h. After electrophoresis, the gel was stained with Coomassie blue R250 (stain buffer, Bio-Rad) for one hour and then destained with a buffer consisting of 10% acetic acid and 20% methanol. The gel was scanned on a Gel Doc XR (Bio-Rad) imaging system for further analysis.

Size exclusion chromatography (SEC) coupled with multi-angle static light scattering (MASLS) analysis of PolyHb solutions—The absolute MW distribution of PolyHb solutions was measured using a SEC column (Ultrahydrogel linear column, 10 μm, 7.8×300 mm, Waters, Milford, Mass.) driven by a 1200 HPLC pump (Agilent, Santa Clara, Calif.), controlled by Eclipse 2 software (Wyatt Technology, Santa Barbara, Calif.) connected in series to a DAWN Heleos (Wyatt Technology) light scattering photometer and an OptiLab Rex (Wyatt Technology) differential refractive index detector. The mobile phase consisted of 20 mM PB (pH 8.0), 100 ppm NaN₃, and 0.2 M NaCl (Fisher Scientific) in HPLC grade water that was filtered through a 0.2 μm membrane filter. PolyHb solutions were diluted to 1 mg/mL with the mobile phase, and 60 μL of sample was injected into the column via a 1200 Autosampler (Agilent). All data were collected and analyzed using Astra 5.3 (Wyatt Technology) software to calculate the weight average molar mass (M_{W}) of the PolyHb solution.

O₂-PolyHb equilibria—The O₂ affinity (P₅₀) and cooperativity coefficient (n) of PolyHb solutions were regressed from O₂-PolyHb equilibrium curves measured on a Hemox Analyzer (TCS Instruments, Southampton, Pa.) at 37° C.

Samples were prepared by thoroughly mixing 100 μL of sample with 5 mL of Hemox buffer (pH 7.4, TCS Instruments), 20 μL of Additive-A, 10 μL of Additive-B and 10 μL of anti-foaming agent. The PolyHb sample was allowed to equilibrate to a pO₂ of 145±2 mm Hg using compressed air. After equilibrating the sample for 45 minutes, the gas stream was switched to pure N₂ to deoxygenate the bHb/PolybHb sample. The absorbance of oxy- and deoxy-Hb in solution was recorded as a function of pO₂ via dual wavelength spectroscopy. O₂-PolyHb equilibrium curves were fit to a four-parameter (A₀, A_{∞}, P₅₀, n) Hill model (Equation 1). In this model, A₀ and A_{∞} represent the absorbance at 0 mm Hg and full saturation, respectively. The cooperativity coefficient is represented by n, and the pO₂ at which the PolyHb is half-saturated with O₂ is represented by the O₂ affinity or P₅₀.

$$Y = \frac{Abs - {A}_{0}}{{A}_{∞} - {A}_{0}} = \frac{{pO}_{2}^{n}}{{pO}_{2}^{n} + {P}_{50}^{n}} \qquad ( 1 )$$

Viscosity and colloid osmotic pressure (COP)—The viscosity of PolyHb solutions was measured in a DV-II Plus rheometer (Brookfield Engineering Laboratories, Middleboro, Mass.) at a shear rate of 150 s⁻¹, whereas the COP was measured using a 4420 colloid osmometer (Wescor, Logan, Utah).

Results for the synthesis of ultrahigh MW T- and R-state PolybHb solutions bHb was polymerized using glutaraldehyde (G) as the cross-linking reagent at a G:Hb molar ratio of 50:1 for T-state PolybHb and 40:1 for R-state PolybHb. After polymerization, each PolybHb mixture was fractionated with a 500 kDa TFF HF cartridge to remove tetrameric Hb and Hb oligomers with MW less than 500 kDa. PolybHb fractions with MW above 500 kDa were characterized in this study.

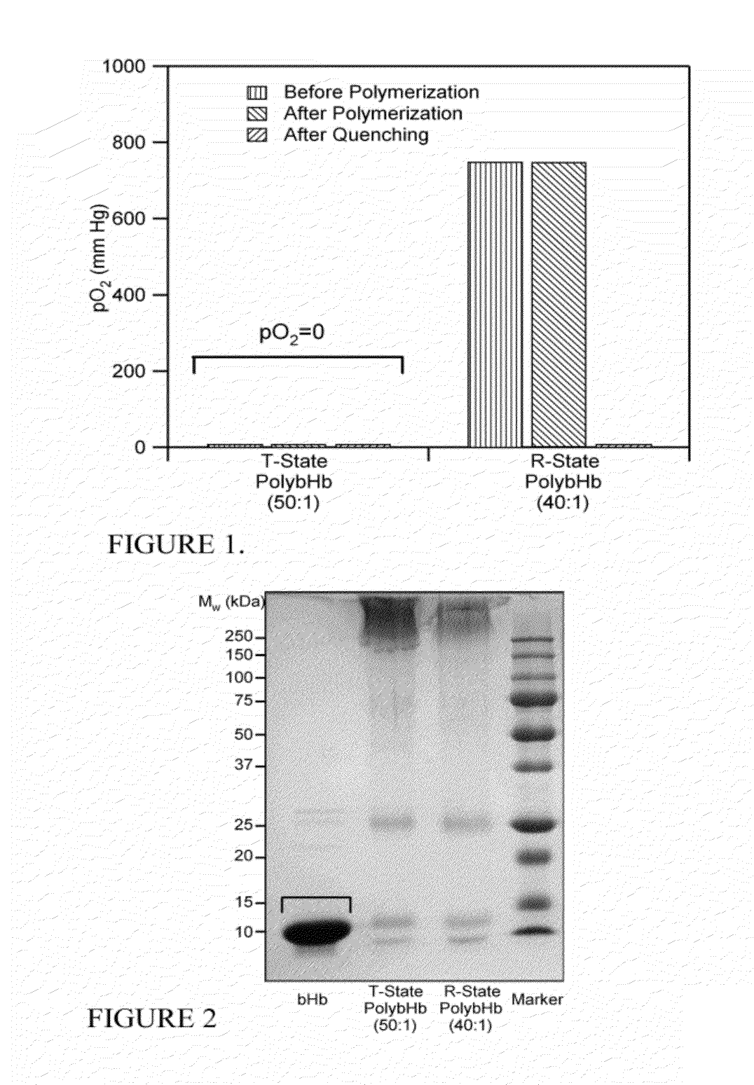

pO₂ of PolybHb solutions—To ensure that PolybHb was in the T- or R-state, the pO₂ at various stages of the bHb polymerization process was measured and shown in FIG. 1. For T-state PolybHb, the pO₂ of the bHb solution was reduced to 0 mm Hg by argon purging and subsequent titration of Na₂S₂O₄ before polymerization and remained at 0 mm Hg after the polymerization reaction and subsequent quenching with reducing agents. These results show that under the protection of an inert atmosphere, bHb was polymerized in an oxygen-free environment and maintained in the deoxygenated state (T-state) during the polymerization process. For R-state PolybHb, the pO₂ before and after polymerization was kept above the measurement range of the oxygen detector. This shows that bHb was saturated with oxygen and bHb was maintained in the R-state. After quenching the R-state bHb polymerization reaction with NaCNBH₃ and NaBH₄, the pO₂ of the R-state PolybHb solution dropped to 0 mm Hg. This pO₂ drop is due to the reduction of O₂ by NaBH₄.

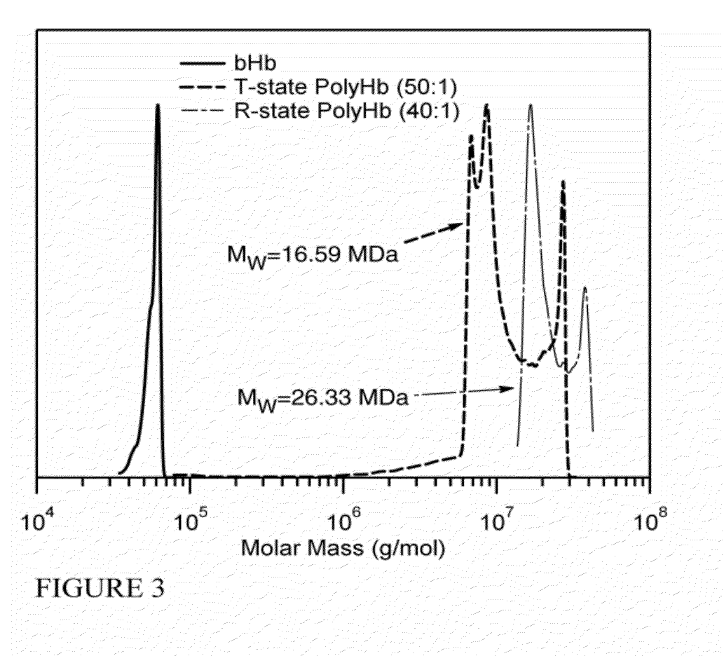

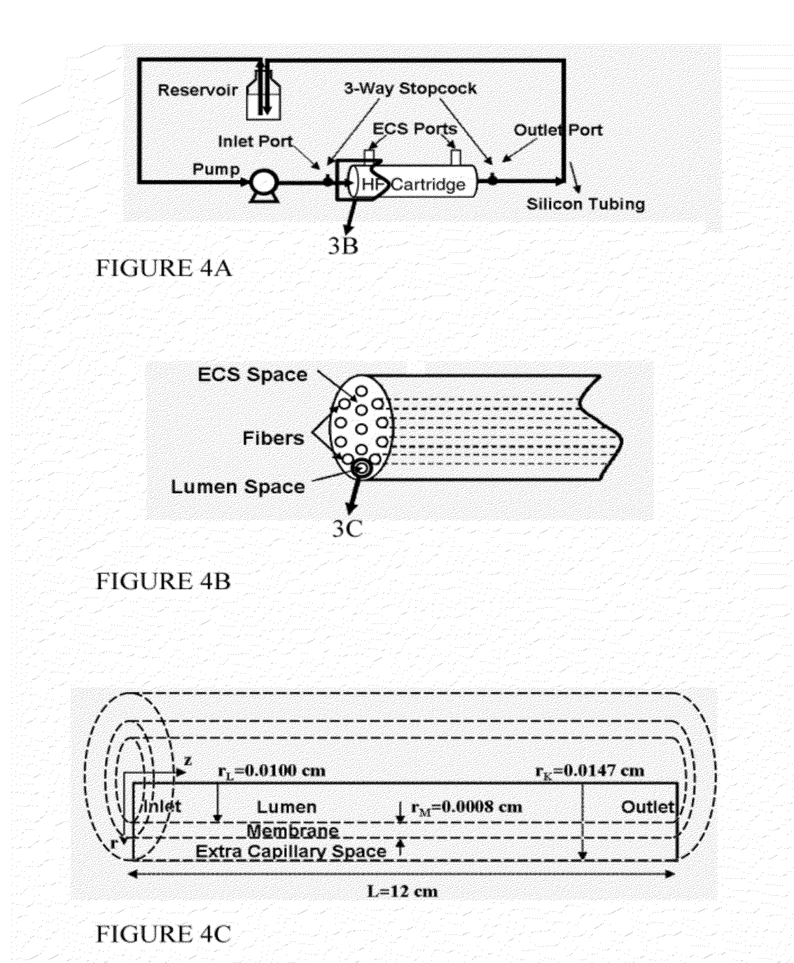

SDS-PAGE and MW distribution of PolybHb solutions—FIG. 2 shows the SDS-PAGE of native bHb and high MW PolybHbs. Both T- and R-state PolybHb show a strong band above 250 kDa and very weak bands around 15 and 30 kDa suggesting that T- and R-state PolybHb mostly consists of inter- and intra-molecular cross-links. Therefore, these results show the presence of very little α and β monomers (these correspond to the two bands around 15 kDa, the lower band represents α subunits while the upper band represents β subunits) as well as α₂/β₂/αβ dimers in solution (these correspond to the band at 30 kDa), confirming that TFF through a 500 kDa HF cartridge was very effective in removing small MW Hb species less than 500 kDa in MW. The majority of both R- and T-state PolybHb solutions was above 250 kDa in MW with the R-state PolybHb being slightly larger (FIG. 2). Light scattering results confirmed that R-state PolybHb has a larger MW distribution compared to T-state PolybHb (Table 1). Despite this difference, both PolybHb solutions possess large MWs ranging from 16.59 to 26.33 MDa (260˜400 bHb tetramers). Light scattering results also indicate that there is no free Hb in the PolybHb solution, since there is no peak corresponding to bHb tetramers (FIG. 3). This shows that all bHb tetramers were polymerized in the reaction. However, the SDS-PAGE results indicates that an extremely small fraction of the PolybHb solutions possess uncross-linked α and β monomers as well as α₂/β₂/αβ dimers. While all the tetrameric Hb is polymerized, there are some α and β monomers as well as αβ/α₂/β₂ dimers that are not cross-linked within the PolybHb superstructure.

P₅₀ and n of PolybHb solutions—The regressed P₅₀ and cooperativity coefficient (n) of bHb and fractionated PolybHb solutions are shown in Table 1. The P₅₀ of R-state PolybHb is approximately 0.3 mm Hg, which is much lower than that of T-state PolybHb which is approximately 41 mm Hg. The cooperativity coefficients of both T- and R-state PolybHb solutions are less than 1.

MetHb level of PolybHb solutions—The metHb level of native bHb, T- and R-state PolybHb is shown in Table 1. The metHb level of native bHb is very low (<1%), since it was purified from fresh bRBCs and stored at −80° C. T- and R-state PolybHb had similar metHb levels, which were both below 4%.

Viscosity and COP of PolybHb solutions—The viscosity and COP of PolybHb solutions is shown in Table 1. The viscosity of both PolybHb solutions was higher than that of bHb. However, the T-state PolybHb solutions displayed lower COP versus R-state PolybHb solutions.

TABLE 1
Biophysical properties of ultrahigh molecular weight 50:1 T- and 40:1 R-state PolybHb solutions.

| | | Tetrameric | | | MetHb | Concentration | Viscosity | COP |
|---|---|---|---|---|---|---|---|---|
| Solution | M_{W} (MDa) | Hb (%) | P₅₀ (mm Hg) | n | Level (%) | (g/dL) | (cp) | (mm Hg) |
| bHb | 0.065 | 100 | 27 | 2.8 | 0.47 | 10 | 1.6 | 38 |
| 50:1 T | 16.59 | 0 | 40 | 0.9 | 3.3 | 10 | 11.4 | 1 |
| 40:1 R | 26.33 | 0 | 1 | 0.7 | 3.4 | 10 | 7.8 | 7 |

Discussion of Results

Biophysical properties of T- and R-state PolybHb solutions—The goal of this study was to synthesize high MW T- and R-state PolyHbs with no tetrameric Hb and large molecular sizes (>500 kDa). bHb was polymerized in distinct quaternary states by carefully controlling the pO₂ of the solution during the polymerization process. In addition, we characterized certain biophysical properties of the PolyHbs.

For T-state PolybHb, the bHb solution was thoroughly deoxygenated before polymerization and polymerized under an inert argon atmosphere. The PolybHb obtained in this manner was maintained in the homogeneous T-state via intra- and inter-molecular glutaraldehyde cross-links. To produce R-state PolybHb, bHb was first transformed into the R-state by completely oxygenating the bHb solution with O₂ and subsequently polymerizing the bHb under an O₂ saturated environment to ensure that the bHb was polymerized in the homogeneous R-state. Therefore, after fractionating the PolybHb mixture with a 500 kDa TFF cartridge, both T- and R-state PolybHb solutions contained no tetrameric Hb. To our knowledge, this is the first time that homogeneous T- and R-state PolybHb solutions were synthesized. The two commercially manufactured PolyHbs, HBOC-201 and PolyHeme®, make no mention of requiring the pO₂ to be lowered to about 0 mm Hg to create 100% T-state PolyHb or raising the pO₂ so as to saturate the Hb to create R-state PolyHb. These commercial products must therefore be considered heterogeneous with respect to the composition of the PolyHb quaternary state. Therefore, our homogeneous T- and R-state PolybHbs can provide a better material for clinical evaluation.

The MW distribution of R-state PolybHb is slightly higher than that of T-state PolybHb even though the polymerization reaction was conducted at a lower G:Hb molar ratio. There are two reasons why this makes sense. First, it has been reported that the reactivity of glutaraldehyde to oxy-Hb is much greater than that to deoxy-Hb. Thus, R-state polymerization can generate bigger aggregates of bHb compared to T-state polymerization. Our results are consistent with that in the literature with respect to this phenomenon. The second reason is due to the presence of Na₂S₂O₄ in the bHb solution. Na₂S₂O₄ was used in the T-state polymerization process to scavenge oxygen from the bHb solution, and therefore maintain the pO₂ at 0 mm Hg. Na₂S₂O₄ can react with free aldehyde groups, thereby quenching some of the glutaraldehyde and reducing the actual G:Hb molar ratio to a level lower than the reported level of 50:1. Hence, reducing the MW of T-state PolybHb compared to R-state PolybHb.

Despite the difference in the MW distribution of our T- and R-state PolybHbs, both of these solutions possess large MWs ranging from 16.59 to 26.33 MDa and no tetrameric Hb in solution (Table 1). Tetrameric Hb was removed from the PolybHb solution by filtering it through a 500 kDa TFF cartridge. The high MW of the PolybHb solutions being free of lower MW PolyHb and tetrameric Hb conveys several advantages over other lower MW compositions.

First, the tetrameric Hb and lower MW components of PolyHb solutions are able to extravasate through the pores in blood vessels or interact more closely with endothelial cells covering the vascular lumen and can scavenge NO from the surrounding endothelial cells or the sub-endothelial compartments. This causes the smooth muscle cells to constrict leading to vasoconstriction in the microcirculation and eventual systemic hypertension. These side-effects can be aggravated in a dose-dependent manner. The high MW T- and R-state PolybHbs synthesized in this study possess no tetrameric Hb and perhaps more importantly exceed 500 kDa in MW and therefore are less likely to have these detrimental side-effects.

Another advantage of the high MW PolyHb compositions is that at concentrations of 10 g/dL, the viscosity of the high MW PolybHb compositions is higher than that of blood (˜3 cp) or lower MW PolyHb compositions as shown in Table 2 in example 2 below. In this study, the viscosity of both PolybHb solutions increases as the PolybHb concentration increases because the molecular interactions between high MW PolybHb molecules are enhanced as the concentration increases in solution. Originally blood substitutes were designed with the assumption that lower blood viscosity is always beneficial. However, blood viscosity directly influences blood vessel diameter due to the shear stress interaction with the endothelium. It is known that a decrease in blood viscosity induces vasoconstriction. Hence, blood viscosity is an important determinant of vasoactivity. PolybHbs can interact mechanically in terms of shear stress, presumably leading to a difference in mechanotransduction with the endothelium. Transfusion of these high viscosity solutions may be advantageous, since these solutions could stimulate NO generation via mechanotransduction of the endothelium. The release of NO would relax the tone of blood vessels and alleviate the vasoconstrictive effect. Additionally, as the high MW PolybHb solutions are proposed to be used during anemic conditions, where blood viscosity is already low, the increase in plasma viscosity induced by the high MW PolybHb will not increase peripheral vascular resistance.

Yet another advantage of the high MW PolyHb is that COP of high MW PolybHb compositions is lower than the COP for bHb and low MW PolyHbs as shown in Table 2 in example 2 below. This is due to two reasons. First, there is no free Hb present in the PolybHb solution. Second, filtering the PolybHb solutions through the 500 kDa TFF cartridge substantially removed Hb species smaller than 500 kDa. The COP of the PolybHb solutions is lower than that of normal blood (27 mm Hg), and can be adjusted to physiological levels by supplementing PolybHb solutions with human serum albumin solution. When transfused, this should enable simultaneous oxygen transport and blood volume expansion. Moreover, oxygen carrying capacity is in principle a direct function of the concentration of functional Hb molecules. The present high MW PolybHb compositions have a highest oxygen carrying capacity due to their extremely low COP. Therefore, these solutions will not elicit autotransfusion and dilute the HBOC concentration in the blood, since their COP is lower than that of blood. As for other commercial HBOCs, augmenting their concentration is not an option in order to increase their oxygen carrying capacity, since they have increased COP. The high COP will promote the flow of interstitial fluid into the circulation, diluting the HBOC, thus lowering the circulating concentration of Hb and increasing the blood volume, a self-limiting process. In contrast, the PolybHbs described in this work constitute a novel set of HBOCs that overcomes these problems.

Another advantage of high MW PolyHbs is that they may exhibit longer circulation times compared to lower MW PolyHbs. It has been shown that the half-life of PolyHbs is proportional to the MW of the PolyHb and reached about 12-15 h for the 192 kDa fraction of Hemolink (Hemosol Corp., Mississauga, Canada) to 20 h for the 576 kDa fraction of HBOC-201. The high MW PolybHbs of the present invention possess MWs 50-fold greater than the 576 kDa fraction of HBOC-201. Thus, it is reasonable to predict that the R-state and T-state high MW PolybHbs of the present invention should display longer circulation times when dosed at equal concentration and volume. Indeed, in vivo experiments showed that R-state PolybHb oxidized faster and to a greater extent than T-state PolybHb. In addition, T-state PolybHb exhibited a longer circulating half-life, slower clearance and longer systemic exposure time compared to R-state PolybHb.

After curve fitting the O₂-PolybHb equilibrium data to the Hill equation, the P₅₀ of both fractionated samples varied greatly (Table 1). The P₅₀ of the high MW T-state PolybHb was around 41 mm Hg similar to the reported value of 38 mm Hg for HBOC-201 (glutaraldehyde polymerized PolybHb manufactured by Biopure Corp., Cambridge, Mass.), suggesting that the T-state PolybHb may have similar oxygen transporting ability to HBOC-201. The P₅₀ of R-state PolybHb is 0.3 mm Hg which demonstrates that cross-linking Hb in the R-state can greatly increase the oxygen affinity of the product in a polymerization-dependent manner. Restitution of the oxygen carrying capacity of low P₅₀ materials compared to normal blood does not affect the total amount of oxygen transported, but affects the amount of oxygen released at each segment of the circulation. Therefore for the purpose of transfusion medicine, a moderate increase in oxygen carrying capacity with decreased oxygen affinity HBOCs should provide more effective oxygen delivery. The O₂ transport capacity of ultrahigh MW T- and R-state PolybHb solutions was investigated during extreme anemia. Restitution of the O₂ carrying capacity with T-state PolybHb exhibited lower arterial pressure and higher functional capillary density compared to R-state PolybHb. Central arterial O₂ tensions increased significantly for R-state PolybHb compared to T-state PolybHb, conversely, microvascular O₂ tensions were higher for T-state PolybHb compared to R-state PolybHb. The increased tissue pO₂ attained with T-state PolybHb results from the larger amount of O₂ released from the PolybHb, maintenance of macrovascular and microvascular hemodynamics compared to R-state PolybHb. These results suggest that the extreme high oxygen affinity of R-state PolybHb prevented O₂ bound to PolybHb from been utilized by the tissues. The results presented here show that T-state PolybHb, a high viscosity O₂ carrier, is an excellent example of an appropriately engineered O₂ carrying solution, which preserves vascular mechanical stimuli (shear stress) that is lost during anemic conditions and reinstates oxygenation, without hypertensive or vasoconstriction responses observed in previous generations of HBOCs. In the case of severe blood loss, clinically referred to as hemorrhagic shock (HS), subsequent small volume resuscitation with hypertonic saline (HTS) followed by infusion of ultrahigh MW T-state PolybHb enhanced tissue oxygenation, without the development of hypertension or vasoconstriction. Furthermore, a mixture of HBOCs with different P₅₀s may provide an even more efficient mechanism for restoring optimal oxygen delivery with a minimal amount of material.

The cooperativity of the two PolybHb solutions is <1 (Table 1) compared to the value for unmodified bHb (2.8). This is due to the intra- and inter-molecular glutaraldehyde cross-links, which freezes the quaternary structure of Hb and reduces its structural flexibility. Therefore, the quaternary structure changes which otherwise would occur during normal oxygen binding/offloading are hindered by the cross-links. This results in a significant loss of cooperative O₂ binding to the Hb tetramer.

The metHb level of both PolybHbs were lower than 4% (Table 1), which fulfill the standard 10% metHb requirement that is frequently cited in the literature. Several steps were taken to retard autoxidation. First, the initial purified Hb had a metHb level lower than 1%. Second, the processes for deoxygenating and oxygenating bHb as well as fractionation of the PolybHb mixture by TFF were all conducted in an ice bath in order to slow down oxidization of the heme. Third, PolybHb solutions were buffer exchanged against modified lactated Ringer's solution containing N-acetyl-L-cysteine, an antioxidant, thereby limiting heme oxidation.

### Example 2

The following is a description of the specific methods used to produce variable MW bovine PolyHb (PolybHb) compositions in the T-state. The PolybHb compositions produced by these methods were then tested in later examples.

Methods used in example 2—The methods described in example 1 were used to polymerize bHb with glutaraldehyde in the T-state at the following molar ratios of glutaraldehyde to bHb: 20:1, 30:1, 40:1 and 50:1. The polymerization reaction was then quenched according to the methods described in example 1.

Separation of PolybHb solution—Initially, each PolybHb solution was clarified by passing it through a glass chromatography column packed with glass wool in order to remove large particles. The glass wool was autoclaved at 250° C. for 30 min before being used to clarify the PolybHb solution, while all tubing, glassware and plasticware were immersed in 1 M NaOH solution for more than 6 hours to degrade any endotoxin present, followed by thorough rinsing with HPLC grade water. The clarified PolybHb solution was then separated into two distinct MW fractions with a 500 kDa TFF cartridge (Spectrum Labs). The retentate mostly contained PolybHb molecules that were larger than 500 kDa, while the filtrate mostly contained PolybHb molecules that were smaller than 500 kDa. The filtrate was subsequently concentrated on a 100 kDa TFF cartridge (Spectrum Labs). Therefore, two distinct MW fractions of PolybHb were obtained after separation of each PolybHb mixture. After separation, the PolybHb solutions were buffered exchanged in a modified Ringer's lactate solution as described in example 1. All PolybHb solutions were then analyzed according to methods described in example 1.

Results for the Synthesis of T-State PolybHb Solutions

The MW distribution of the low MW PolybHb fractions (<500 kDa) did not increase significantly at cross-link densities ranging from 20:1 to 50:1 (Table 2). However, all PolybHb fractions were larger than that of a Hb tetramer. On the other hand, high MW PolybHb fractions (>500 kDa) exhibited an increase in MW as the cross-link density increased from 20:1 to 50:1. High MW PolybHbs did not show the presence of tetrameric Hb.

Therefore this method yields the high MW fraction of PolybHb (i.e., MW greater than 500 kDa in size) that is free of tetrameric Hb as well as the low MW fraction that is less than 500 kDa in size (Table 2).

Both the high and low MW fractions of PolybHb exhibited low metHb levels (<5%); however, high MW PolybHbs exhibited lower metHb levels than low MW PolybHbs (Table 2). The high MW PolybHb resulted in lower metHb levels, regardless of cross-linking density.

High MW PolybHbs exhibited increased solution viscosity and decreased colloid osmotic pressure (COP) as the cross-link density increased from 20:1 to 50:1 (Table 2). However, low MW PolybHb solutions exhibited similar solution viscosities independent of cross-link density, whereas the COP decreased as the cross-link density increased.

The regressed P₅₀ and cooperativity coefficient (n) of fractionated PolybHb solutions are presented in Table 2. Polymerization of Hb in the T-state resulted in a left shift in the O₂-PolyHb equilibrium curve. The P₅₀ of low MW PolybHb solutions decreased slightly as the cross-link density increased. However, the P₅₀ of high MW PolybHb solutions was independent of cross-link density. The cooperativity of PolybHb solutions above and below 500 kDa in size decreased with increasing cross-link density. It also should be noted that PolybHb fractions above and below 500 kDa in size separated from the same reaction mixture possessed similar cooperativity coefficients.

TABLE 2
Biophysical Properties of T-state PolybHbs.

| | | | | | | |
|---|---|---|---|---|---|---|
| PolyHb | Viscosity^{#} | COP^{γ} | P₅₀ | | MetHb | M_{W} |
| 10 g/dL | (cp) | (mm Hg) | (mm Hg) | n | Level (%) | (kDa) |

Below 500 kDa in size

| | | | | | | |
|---|---|---|---|---|---|---|
| 20:1 | 1.4 | 50 | 34.6 | 1.2 | 2.5 | 114.6 |
| 30:1 | 1.4 | 48 | 28.6 | 1.0 | 2.0 | 101.7 |
| 40:1* | 1.0 | 18 | 27.7 | 0.8 | 3.6 | 180.9 |
| 50:1** | 1.0 | 18 | 23 | 0.8 | 2.8 | 166.2 |

Above 500 kDa in size

| | | | | | | |
|---|---|---|---|---|---|---|
| 20:1 | 1.7 | 35 | 30.5 | 1.2 | 1.3 | 655.7 |
| 30:1 | 1.8 | 28 | 33.8 | 1.0 | 1.9 | 723 |
| 40:1 | 7.2 | 5 | 38.1 | 0.9 | 2.1 | 1360 |
| 50:1 | 11.4 | 1 | 34.9 | 0.8 | 2.0 | 23710 |

*5.6 g/dL;
**4.2 g/dL;
^{#}Measured at a shear rate of 150 s⁻¹ at 37° C.;
^{γ}Colloid osmotic pressure (COP) measured at 25° C.; P₅₀ (oxygen affinity, pO₂ at which the HBOC is half-saturated with oxygen); n (cooperativity coefficient); metHb Level (fraction of Hb in which the heme iron is in the 3 + valence state); M_{W} (weight average molar mass).

Discussion of Results

Biophysical properties of T-state PolybHb solutions—In order to minimize oxidation of the heme, bHb was polymerized in an anoxic environment (pO₂=0 mm Hg) and quenched with the strong reducing agent NaBH₄. As a further precaution against auto-oxidation of the heme, fractionated PolybHb solutions were buffer exchanged against modified Lactated Ringer's solution containing N-acetyl-L-cysteine, an antioxidant, which limits heme oxidation. Compared with the high MW PolybHb fraction, the low MW PolybHb fraction displayed higher MetHb levels. This result is expected. Since free tetrameric Hb (α₂β₂) exists in equilibrium with αβ dimers, the low molecular weight PolybHb fraction contains more dimers in solution, which are more prone to oxidation versus their tetrameric counterpart. This gives rise to the higher metHb levels of low MW PolybHbs versus high MW PolybHbs.

At low cross-link densities (20:1 and 30:1), the MW distribution shows that the PolybHb fractions were polymerized primarily by intramolecular cross-links with a very minor portion possessing intermolecular cross-links. Under these conditions, the intramolecular cross-linking sites on the bHb molecule are not fully saturated with cross-linker. On the other hand at higher cross-link densities (40:1 and 50:1), it is likely that the majority of intramolecular cross-linking sites were saturated, which facilitates subsequent intermolecular cross-linking between adjacent Hb tetramers in solution.

The polymerization process yields larger sized particles that can interact strongly with each other, especially when the PolybHb solution is concentrated via TFF. Hence, polymerization of bHb yields high MW fractions that display an increase in viscosity with increasing cross-link density. The low MW fractions do not display such a trend, because the polymerized species are much smaller in size and do not interact as strongly with each other, and as the cross-link density increases there is very little PolybHb in solution. Conversely, the COP is inversely related to particle size. Therefore, all PolybHb fractions below 500 kDa in MW exhibited similar COPs. However, the COP decreased as the cross-link density increased for PolybHb fractions more than 500 kDa in MW, this is due to the concomitant increase in MW of the PolybHb molecules with increasing cross-link density.

After curve fitting the O₂-PolybHb equilibrium data to the Hill equation, the fractional saturation of the PolybHbs at a pO₂ of 145 mm Hg varied among the different fractionated samples. Before polymerization, the pO₂ of the initial bHb solution was maintained at 0 mm Hg. Therefore, all bHb molecules in solution were maintained in the low oxygen affinity T-state. After polymerization of T-state bHb, the reducing agent NaBH₄ was added to the reaction to quench any remaining glutaraldehyde in solution, as well as to reduce the resulting Schiff bases. Hence, T-state PolybHb is frozen in the low oxygen affinity state via intra- and inter-molecular glutaraldehyde cross-links within the bHb tetramer and between tetramers. For T-state PolybHb solutions above 500 kDa, the P₅₀'s were much higher than that of unmodified bHb (27 mm Hg) and ranged from 31.1-37.3 mm Hg across cross-link densities ranging from 20:1 to 50:1, which was very close to the reported value of 38 mm Hg for Hemopure® (glutaraldehyde cross-linked bovine Hb manufactured by Biopure Corp., Cambridge, Mass.). The cooperativity of all PolybHb solutions is much less compared to the value for unmodified bHb (2.8). This is caused by the glutaraldehyde cross-links present within and between the globin chains, which restrict transmission of any quaternary changes in the Hb structure to other neighboring globin chains within the Hb tetramer. This results in a significant loss of cooperative binding of O₂ molecules to the Hb tetramer.

These materials were evaluated in a 10% top-load model in order to analyze systemic and microvascular responses to the MW and plasma concentration of T-state PolybHb solutions. Infusion of PolybHb solutions with MWs below 500 kDa elicited significant hypertension and vasoconstriction that was proportional to the plasma concentration, and independent of PolybHb cross-link density. Additionally, infusion of PolybHb solutions with MWs above 500 kDa elicited hypertension and vasoconstriction that was proportional to the plasma concentration and inversely proportional to the PolybHb cross-link density. Thus, highly cross-linked T-state PolybHb solutions with MWs above 500 kDa do not appear to have detrimental vascular side-effects to the same extent as lower MW PolyHbs. These data support the concept that HBOC size/molecular weight influences its proximity to the vascular endothelium and molecular diffusivity.

### Example 3

The following is a description of the specific methods used to produce variable MW bovine PolyHb (PolybHb) and human PolyHb (PolyhHb) compositions in the T- and R-states. The main difference between the method described in this example and the methods described in examples 1 and 2 is the absence of any column chromatography step for buffer exchange. In this description, separation of the PolyHb mixture and buffer exchange in the modified Ringer's lactate solution is achieved via diafiltration using a 500 kDa TFF cartridge. Therefore, all the PolyHbs in this example are more than 500 kDa in MW. The PolyHb compositions produced by these methods were then tested in later examples.

Hb Purification—In this example, bovine Hb and human Hb were purified from RBCs according to the methods described in example 1. However, human RBCs were obtained from the Columbus, Ohio branch of the American Red Cross.

Hb Polymerization—Bovine Hb was polymerized with glutaraldehyde and quenched according to the methods described in example 1 in both the T- and R-states at glutaraldehyde to bHb molar ratios of 20:1 and 30:1. Human Hb was polymerized with glutaraldehyde and quenched according to the methods described in example 1 in both the T- and R-states at the following glutaraldehyde to hHb molar ratios: (T-state: 40:1 and 50:1; R-state: 20:1 and 30:1).

Diafiltration of PolyHb—Both T- and R-state PolyHb solutions were first passed through a column packed with glass wool to remove any large particles from solution. The clarified PolyHb solution was then subjected to 4 cycles of diafiltration on a 500 kDa MW cutoff TFF cartridge (Spectrum Labs) with a modified lactated Ringer's buffer (NaCl 115 mM, KCl 4 mM, CaCl₂ 1.4 mM, NaOH 13 mM, sodium lactate 27 mM, and N-acetyl-L-cysteine 2 g/L). At the last stage of diafiltration, the PolyHb solution was concentrated and then stored at −80° C. until needed.

All PolyHb solutions were then analyzed according to the methods described in example 1.

Results for the Synthesis of T- and R-State PolybHb and PolyhHb Solutions

Polymerization of Hb—The major reaction in the polymerization of Hb by glutaraldehyde involves Michael addition between α,β-unsaturated oligomeric aldehydes and primary amine groups on lysine residues that are present on the surface of Hb. Some Schiff bases form during this polymerization step, but they are all reduced by adding sodium borohydride or sodium cyanoborohydride at the end of the polymerization reaction. Therefore, polymerization of Hb is based on stable C—N bonds that will not hydrolyze in aqueous solution.

pO₂ of Hb solutions during the polymerization process—For the preparation of T-state bovine and human PolyHbs, the pO₂ of Hb solutions was maintained at 0 mm Hg at all stages of the polymerization process, while it remained above the measurement range of the blood gas analyzer during the polymerization of R-state PolyHbs. These results indicate that Hb was kept exclusively in either the T- or R-state during the polymerization reactions. The pO₂ of R-state PolyHbs solutions dropped to 0 mm Hg after quenching. This is due to the displacement of dissolved O₂ in solution by the H₂ gas generated from NaBH₄, which was used to quench the polymerization reaction.

Absolute MW distribution of PolybHb solutions—Light scattering results indicate the absence of free Hb in all PolyHb solutions, since no corresponding individual α/β subunit, α₂/β₂/αβ dimer, or bHb tetramer peak was observed. The M_{W} of bovine and human PolyHbs is listed in Tables 3 and 4, respectively. It is evident that the M_{W} of PolyHbs increased with increasing cross-link density, while R-state bovine and human PolyHbs possessed higher M_{W} than that of the corresponding T-state PolybHbs at the same cross-link density.

Oxygen affinity and cooperativity of PolyHbs—All PolyHbs exhibited hyperbolic-shaped equilibrium curves, which indicate a substantial loss in cooperativity. The O₂-T-state PolyHb equilibrium curves are increasingly shifted to the right of the O₂-Hb equilibrium curve with increasing cross-link density. In contrast, the O₂-R-state-PolyHb equilibrium curves are increasingly shifted to the left of the O₂-Hb equilibrium curve with increasing cross-link density. The dependence of regressed P₅₀ and cooperativity coefficient (n) of bovine and human PolyHb solutions on cross-link density are shown in Tables 3 and 4 with native Hb as the control. The P₅₀s of T-state PolyHbs are higher than that of native Hb and increase with increasing cross-link density. In contrast, all of the R-state PolyHbs display extremely low P₅₀s compared to both native Hb and T-state PolyHbs, which decrease with increasing cross-link density. The cooperativity coefficients (n) of both T- and R-state PolyHbs are lower than that of native Hb.

MetHb level of PolyHb solutions—Tables 3 and 4 list the metHb level of native Hb, T- and R-state PolyHb solutions. The metHb level of native Hb is extremely low (less than 1%), while T- and R-state PolyHb solutions exhibited metHb levels below 6%.

Viscosity and COP of PolyHb solutions. Tables 3 and 4 list the viscosity and COP of both T- and R-state PolyHb solutions with native Hb as the control. At or near a total protein concentration of 10 g/dL, the viscosity of both T- and R-state PolyHb solutions are higher than that of native Hb and increased with increasing cross-link density. In contrast, the COP of PolyHb solutions decreased with increasing cross-link density. However, T-state PolyHb solutions exhibited lower COPs compared to R-state PolyHb solutions when compared at the same cross-link density.

TABLE 3
Biophysical properties of bHb/PolybHb solutions.

| | | | | MetHb | Concentration | Viscosity | COP |
|---|---|---|---|---|---|---|---|
| Solution | M_{W} (kDa) | P₅₀ (mm Hg) | n | Level (%) | (g/dL) | (cp) | (mm Hg) |
| bHb | 65.3 ± 0.6 | 27.37 ± 1.57 | 2.84 ± 0.081 | 0.47 ± 0.099 | 10 | 1.6 | 38 |
| 20:1 T | 749 ± 43 | 37.10 ± 0.94 | 1.05 ± 0.098 | 1.95 ± 0.24 | 10 | 4.8 | 24 |
| 30:1 T | 1330.3 ± 159 | 41.16 ± 3.05 | 1.01 ± 0.015 | 2.90 ± 1.45 | 10 | 14.8 | 2 |
| 20:1 R | 1025.9 ± 122.8 | 2.18 ± 0.90 | 0.56 ± 0.047 | 4.54 ± 1.02 | 10 | 3.6 | 39 |
| 30:1 R | 6256 ± 886.7 | 1.84 ± 0.78 | 0.69 ± 0.11 | 4.50 ± 0.98 | 10 | 9.8 | 14 |

The error bars represent the standard deviation from triplicate reactions.

TABLE 4
Biophysical properties of native hHb/PolyhHb solutions.

| | | | | MetHb | Concentration | Viscosity | COP |
|---|---|---|---|---|---|---|---|
| Solution | M_{W} (kDa) | P₅₀ (mm Hg) | n | Level (%) | (g/dL) | (cp) | (mm Hg) |
| hHb | 62.29 ± 1.63 | 13.27 ± 0.55 | 2.59 ± 0.12 | 0.88 ± 0.33 | 10 | 1.6 | 38 |
| 40:1 T | 3680 ± 1330 | 37.19 ± 1.49 | 0.81 ± 0.05 | 4.32 ± 0.41 | 9.9 | 8.7 | 4 |
| 50:1 T | 18440 ± 7230 | 48.84 ± 4.22 | 0.87 ± 0.16 | 4.99 ± 1.26 | 10 | 9.6 | 4 |
| 20:1 R | 1100 ± 330 | 1.45 ± 0.14 | 0.70 ± 0.06 | 2.97 ± 0.97 | 9.1 | 3.2 | 37 |
| 30:1 R | 5540 ± 2000 | 0.76 ± 0.50 | 0.91 ± 0.51 | 5.81 ± 3.51 | 9.6 | 6.6 | 24 |

The error bars represent the standard deviation from triplicate reactions.

Discussion of Results

This study was conducted to investigate the effect of Hb quaternary state and cross-link density on the biophysical properties of PolyHb solutions.

The MW of a HBOC is an important parameter to consider when evaluating its efficacy and safety for a particular clinical application. PolyHbs with MWs greater than 500 kDa can be used for transfusion applications and should not elicit substantial vasoconstriction and hypertension upon transfusion.

These materials were transfused into animals with moderate anemia. Upon infusion, higher cross-link density PolybHbs did not elicit a significant change in mean arterial pressure and heart rate compared to baseline. In addition, higher cross-link density PolybHbs exhibited less vasoconstriction and higher functional capillary densities compared to lower cross-link density PolybHbs. Interestingly, T-state PolybHbs exhibited less vasoconstriction and hypertension compared to R-state PolybHbs at the same cross-link density.

In vivo experiments also showed that R-state PolybHb oxidized faster and to a greater extent than T-state PolybHb at the same cross-link density. In addition, T-state PolybHb exhibited a longer circulating half-life, slower clearance and longer systemic exposure time compared to R-state PolybHb at the same cross-link density. Within the same quaternary state, it was observed that the higher cross-link density PolybHb exhibited longer half-life versus the lower cross-link density PolybHb.

Interestingly, both T- and R-state PolybHbs also have the potential to act as nitrite reductases and are thus able to reduce nitrite into NO. This suggests that co-administration of T- or R-state PolyHb along with a nitrite salt could reduce vasoconstriction and systemic hypertension upon transfusion of PolyHb solutions.

The O₂ affinity and cooperativity coefficient of T-state PolyHb solutions are low compared to Hb. This is attributed to the ability of glutaraldehyde to react with a wide-range of amino acid residues on Hb, including primary amine groups on lysines and N-terminal valines, sulfhydryl groups on cysteines, imidazole rings on histidines, and phenolic rings on tyrosines. Therefore as the cross-link density increases, there are more amino acid side chains that will be covalently linked to free aldehyde groups on glutaraldehyde. Thus, the intra- and inter-molecular cross-links freeze the quaternary structure of Hb and reduce its structural flexibility in a concentration dependent manner. Hence, the quaternary structure changes which otherwise would occur during normal O₂ binding/offloading are hindered by the presence of the chemical cross-links. This leads to a loss of cooperativity of all PolyHbs compared to native Hb. In contrast to T-state PolyHbs, all R-state PolyHb solutions displayed high O₂ affinities. It was observed that the O₂ affinity increased proportionally with the cross-link density.

All the T- and R-state PolyHb solutions in this study exhibited metHb levels lower than 6%, which satisfy the 10% metHb criterion necessary for transfusion. Interestingly, the metHb level of all PolyHb solutions in our study is not sensitive to the cross-link density. This is ascribed to the preventative measures that were taken to reduce the metHb level during the polymerization process and to retard the autoxidation of heme during the diafiltration process. The overall metHb levels of R-state PolyHb solutions are a little higher than that of T-state PolyHb solutions. This makes sense since the R-state PolyHbs were prepared in an O₂ saturated environment, which made the heme groups more vulnerable to autoxidation.

At a Hb concentration (10 g/dL), close to physiological levels (14 g/dL), the viscosity of both T- and R-state PolyHbs increased with increasing cross-link density and displayed higher viscosities than native Hb (1.6 cp) or blood (˜3 cp). The increase in viscosity with increasing cross-link density is consistent with the increased MW of the PolyHb macromolecule.

High viscosity HBOCs are beneficial in blood transfusion for at least 2 reasons. First, transfusion of high viscosity solutions improves the functional capillary density, which primarily determines in vivo survival after transfusion. Transfusion of high viscosity solutions increases blood vessel wall shear stress, which induces the endothelium to produce the vasodilator nitric oxide (NO) which will thereby alleviate vasoconstriction and hypertension elicited by NO scavenging of HBOCs. Second, high viscosity solutions retard the diffusion of the PolyHb macromolecule in the blood to reduce both the concentration of PolyHb near the vessel wall and facilitated diffusion of excess O₂ to the vessel wall.

The COP is another vital property that a HBOC must possess in order to assess its suitability for transfusion. COP determines the fluid balance between intra- and extravascular space. The COP of both T- and R-state PolyHbs in our study decreased with increasing cross-link density. This is because the COP depends on the number of colloid particles in solution. Therefore at same total protein concentration in solution, the number of PolyHb particles present in solution is inversely proportionally to the PolyHb MW. Although transfusion solutions with higher COPs were considered to cause the in-flow of fluid from the tissue space into intravascular compartment, transfusion of hyperoncotic and hyperviscous HES in hemorrhagic shock could achieve rapid recovery of the microcirculation by increasing blood volume in a short time frame while maintaining cardiac output. Therefore these solutions could be used in oxygenating the hypoxic tissues of patients suffering from hemorrhagic shock.

### Example 4

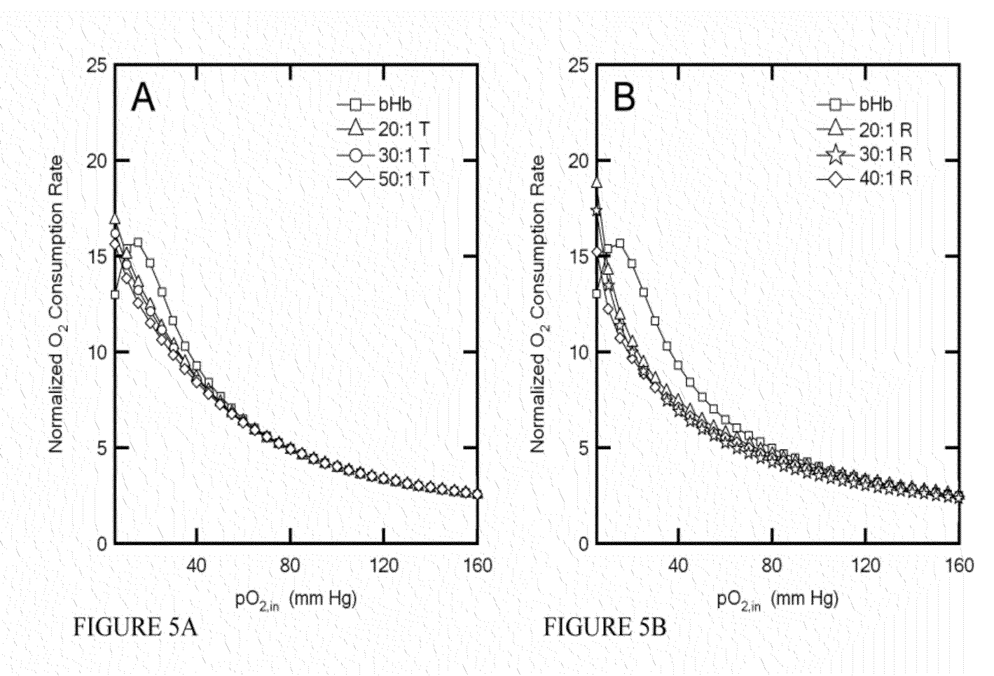

In order to evaluate the ability of T- and R-state PolyHbs synthesized in examples 1 and 3 to potentially oxygenate tissues in vivo and cell cultures in vitro an O₂ transport model was used to simulate O₂ transport in a hepatic HF bioreactor. For this example, an O₂ transport model was developed based on the geometry of a single HF contained in a hepatic HF bioreactor which mimics the in vivo capillary/liver sinusoid structure (FIG. 4). In this geometry, cell culture media containing Hb/PolyHb solution flows through the lumen of the HF bioreactor to provide nutrients to cells (in this case hepatocytes) which reside in the ECS, while simultaneously carrying away metabolic waste products. The cell culture media recirculates throughout the entire HF bioreactor system. The HF membrane has a MW cut-off of 35 kDa and therefore confines Hb/PolyHbs (MW>64 kDa) within the lumenal space of the HF without directly contacting cells cultured within the ECS.

Model Description

The velocity profile in each of the three subdomains (lumen, membrane, and ECS) is calculated from a set of momentum transport partial differential equations (PDEs), shown in Equation (2).

Navier-Stokes Equation (lumen):

$$( \overline{{v}^{′}} \cdot \overline{∇} ) \overline{{v}^{′}} = - [ \frac{μ}{{l}_{0} {v}_{0} ρ} ] \overline{∇} {P}^{′} + [ \frac{μ}{{l}_{0} {v}_{0} ρ} ] {∇}^{2} \overline{{v}^{′}} \qquad ( 2 ) \text{-} A$$

Brinkman's Equation (membrane and ECS):

$$[ \frac{{l}_{0}^{2}}{κ} ] \overline{{v}^{′}} = \overline{∇} ( - {P}^{′} ) + {∇}^{2} \overline{{v}^{′}} \qquad ( 2 ) \text{-} B$$

Where

$$\overline{{v}^{′}} = \frac{\overline{v}}{{v}_{0}} , {P}^{′} = \frac{P}{μ {v}_{0} / {l}_{0}} \cdot \overline{{v}^{′}}$$

is the dimensionless velocity vector and P′ is the dimensionless pressure. l₀, ν₀, ρ and μ represent the reference length, reference velocity, fluid density and viscosity, respectively.

The mass conservation equations which describe transport of dissolved O₂, total HBOC and O₂-HBOC in dimensionless form are shown in Equation (3). C′ can either represent the dimensionless O₂ partial pressure (pO₂), total HBOC concentration or O₂-HBOC concentration. Co can either represent the reference O₂ partial pressure, total HBOC concentration or O₂-HBOC concentration. D can either represent the diffusivity of O₂ or HBOC. R represents the rate of formation of O₂/O₂-HBOC. The HBOC diffusivity was estimated from the M_{W} using Equation (4).

$$\begin{matrix}\overline{∇} \cdot ( - \overline{∇} {C}^{′} ) = [ \frac{{l}_{0}^{2}}{D} \cdot \frac{R}{{C}_{0}} ] - [ \frac{{v}_{0} {l}_{0}}{D} ] \overline{{v}^{′}} \cdot \overline{∇} {C}^{′} & ( 3 ) \\ D = 1.013 \times {10}^{- 4} {M}_{W}^{- 0.46} ( \frac{{cm}^{2}}{s} ) & ( 4 )\end{matrix}$$

The reaction between O₂ and HBOC is described by Equation (5), where m is the number of O₂ binding sites on a single HBOC molecule. Given the thermodynamic relationship describing the equilibrium between HBOC and O₂ (Equation (6), where a₁-a₄ are the Adair constants), the rate of formation of O₂ (R_{O}_{₂} (mm Hg/s)) or O₂-HBOC (R_{oxyHBOC} (mol/m³*s)) in the lumen is shown in Equation (7). S is defined as the HBOC saturation, i.e. the molar fraction of HBOC that is saturated with O₂ and S_{eq} is the HBOC saturation fraction at equilibrium. [O₂] is the concentration of dissolved O₂, [HBOC] is the concentration of total HBOC and α is the solubility of O₂ in aqueous media. The O₂ consumption rate in the ECS is described by Michaelis-Menten (M-M) kinetics.

$$\begin{matrix}{mO}_{2} + H B O C {⇄}_{{k}^{-}}^{{k}^{+}} oxy H B O C & ( 5 ) \\ {S}_{eq} = \frac{{a}_{1} \cdot {pO}_{2} + 2 {a}_{2} \cdot {pO}_{2}^{2} + 3 {a}_{3} \cdot {pO}_{2}^{3} + 4 {a}_{4} \cdot {pO}_{2}^{4}}{4 ( 1 + {a}_{1} \cdot {pO}_{2} + {a}_{2} \cdot {pO}_{2}^{2} + {a}_{3} \cdot {pO}_{2}^{3} + {a}_{4} \cdot {pO}_{2}^{4} )} & ( 6 ) \\ {R}_{oxyHBOC} = - \frac{α}{m} \cdot {R}_{{O}_{2}} = {k}^{-} \cdot [ H B O C ] ( \frac{{S}_{eq}}{1 - {S}_{eq}} ( 1 - S ) - S ) & ( 7 )\end{matrix}$$

The coupled set of PDEs was solved by the finite element method in Comsol Multiphysics (COMSOL, Inc., Burlington, Mass.) yielding numerical solutions.

This O₂ transport model was used to simulate O₂ transport within a single HF of a hepatic HF bioreactor, in which the circulating cell culture media was supplemented with Hb/PolyHbs. The case where no HBOC was present in the cell culture media served as the control, and all results were normalized on this basis. The oxygenation potential of native Hb in the HF bioreactor was also simulated for comparison.

Results of Simulations

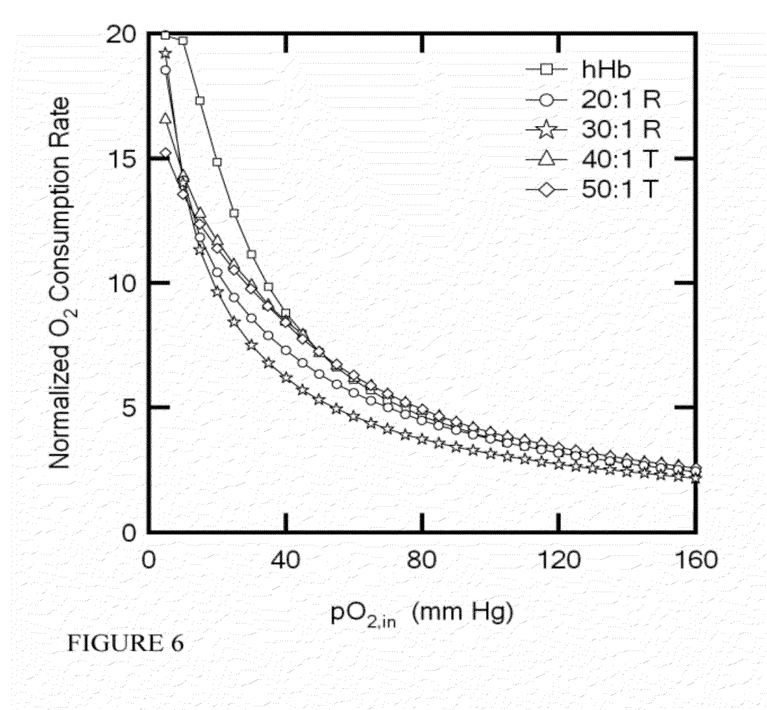

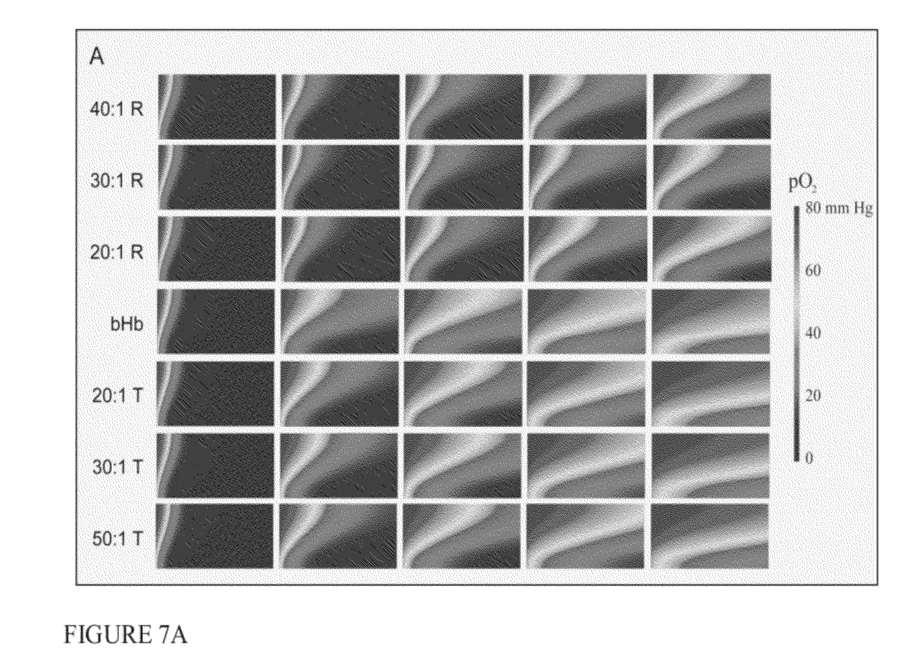

The normalized oxygen consumption rate of C3A hepatocytes cultured in a HF bioreactor with media supplemented with Hb/PolyHb is shown in FIGS. 5 and 6 as a function of inlet pO₂(pO₂ᵢₙ). The value of the normalized O₂ flux monotonically decreased as the pO_{2,in} increased for all HBOCs studied. At low pO_{2,in}s (<40 mm Hg), the oxygen consumption rate of either T- or R-state PolyHb supplementation was lower than that of native Hb, and decreased with increasing cross-link density. In contrast, at higher pO_{2,in}s (˜150 mm Hg), the O₂ consumption rate for bHb supplemented HF bioreactors was similar to that with either T- or R-state PolyHb supplementation at all cross-link densities.

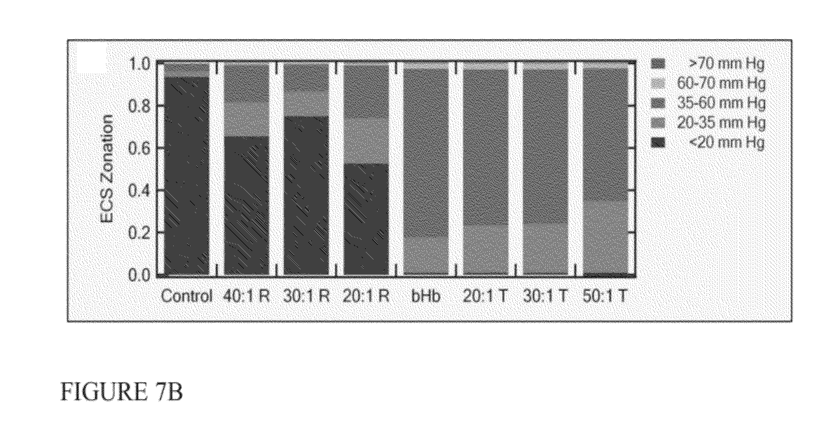

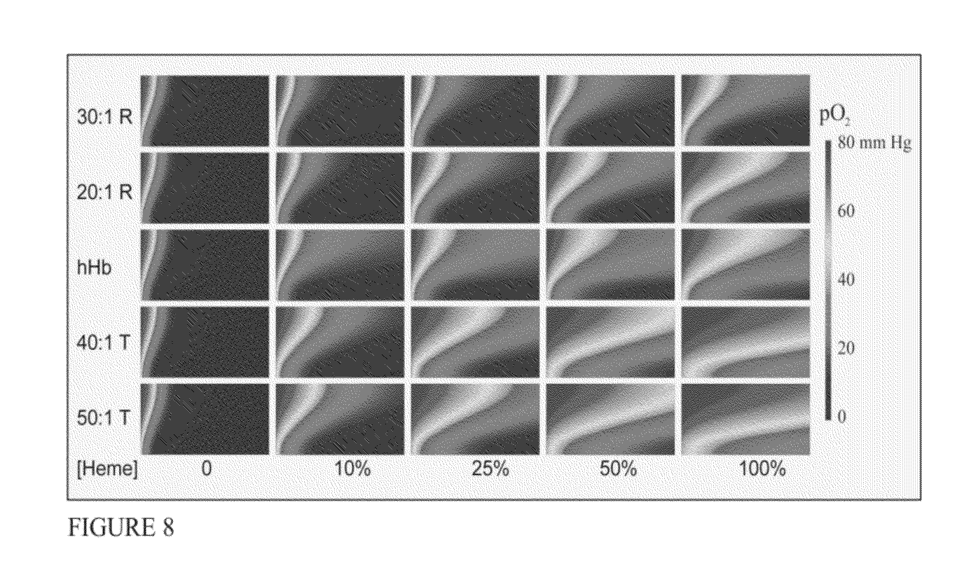

FIGS. 7A and 8 show the pO₂ profiles within a single HF (including lumen, membrane and ECS) under varying concentrations of PolyHb. In this work, the heme concentration was normalized by the average physiological heme concentration in human blood (8800 μM). Each unit in the figure represents a cross-sectional view of a single HF (FIG. 4C). The HF centerline is represented by the top horizontal boundary, while the inlet and outlet of the lumen are represented by the left and right boundaries, respectively. In the absence of Hb/PolyHb in the circulating cell culture media, the majority of space within the HF is hypoxic, with the pO₂ level generally below 20 mm Hg. Oxygenation of the HF bioreactor gradually improves with an increase in PolyHb concentration. Our data shows that T-state PolyHbs with different cross-link densities exhibited similar O₂ transport capabilities compared to Hb, which substantially reduced the volume of the hypoxic space in the HF. In contrast, R-state PolyHbs supported a much lower potential to oxygenate the HF even at a heme concentration of 100%.

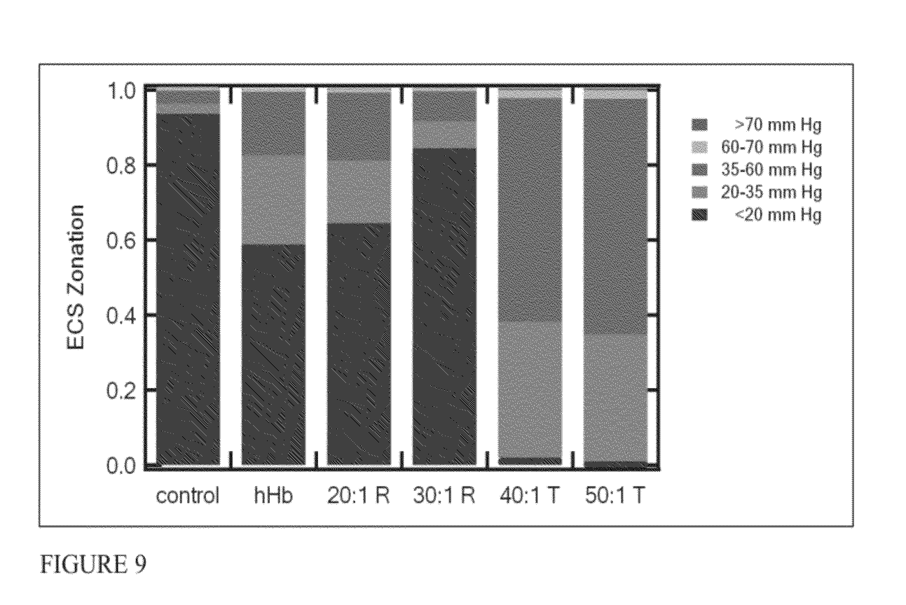

These results are consistent with the ECS zonation plot, which is designed to show further details of the distribution of O₂ within the cell-containing ECS (FIGS. 7B and 9). Oxygenation within the ECS was quantified into the following pO₂ zones: hyperoxic (>70 mm Hg), periportal (60-70 mm Hg), pericentral (35-60 mm Hg), perivenous (20-35 mm Hg) and hypoxic (<20 mm Hg) zones. As mentioned previously, the hypoxic region (pO₂<20 mm Hg) dominates ˜95% of the ECS (˜95%) without supplementation of Hb/PolyHbs and only a small percentage of hepatocytes (˜5%) are subjected to in vivo pO₂ levels (ranging between 20-70 mm Hg). It is clearly apparent that T-state PolyHbs greatly enhanced O₂ transport inside the ECS. In the presence of T-state PolyHbs, the entire ECS volume is in the pO₂ range 20-70 mm Hg and the volume of the hypoxic region diminishes significantly, which is similar to the effect of Hb. In contrast, a large percentage of the volume in the ECS remained hypoxic with supplementation of R-state PolyHbs in the circulating cell culture media. In fact, the volume of the ECS exposed to hypoxic pO₂ levels (<20 mm Hg) increased with increasing cross-link density.

Discussion of Results

Compared to native Hb, all PolyHbs showed slightly lower O₂ consumption rate at lower inlet pO₂s (<40 mm Hg). This is probably due to the low cooperativities and diffusivity of these macromolecules. However, there is not much difference between all PolyHb solutions and native Hb in terms of the normalized O₂ consumption rate at higher inlet pO₂s (˜150 mm Hg). This is consistent with the pO₂ profile in the HF and ECS zonation. In general, the hypoxic region in the ECS shrinks as the P₅₀ of the PolyHb increases in magnitude. We observed that T-state PolyHbs (high P₅₀) enhanced O₂ transport into the ECS of the HF bioreactor in a similar manner compared to Hb. However, under the simulated conditions studied (inlet pO₂=80 mm Hg) the size of the pericentral zone (35-60 mm Hg) decreased with increasing cross-link density, while the size of the perivenous zone (20-35 mm Hg) increased with increasing cross-link density, suggesting that the oxygenation capabilities of T-state PolyHbs doesn't strictly increase with increasing P₅₀. This may be because the ultrahigh MW of PolyHb molecules limit their diffusion in solution, which in turn reduces the amount of O₂ released to the HF ECS. Therefore, this suggests that in order to obtain optimum oxygenation of tissues not only P₅₀ but also molecular size should be taken into account to get a balance between these two properties. However, for R-state PolyHbs which have very high O₂ affinities, the O₂ consumption rate is higher than that of native Hb and T-state PolyHbs only under extremely hypoxic conditions in which the pO₂ level is close to zero, suggesting that R-state PolybHbs cannot offload their store of O₂ until extensive hypoxia occurs. Hence, R-state PolybHbs may be useful in oxygenating hypoxic tissues under extreme duress.

These data suggest that PolyHb supplementation in cell culture media will improve O₂ delivery to cells grown in a cell culturing system. Improved O₂ gradient zonation can improve cell proliferation and function, especially in a HF bioreactor type of device.

What is claimed is:

1. A hemoglobin-based oxygen carrier (HBOC) composition comprising a polymerized hemoglobin (PolyHb) wherein a majority of the hemoglobin (Hb) in the PolyHb is locked in at least one of the tense or relaxed quaternary state and a majority of the PolyHb has a molecular weight (MW) of at least about 500 kDa.

2. The composition of claim 1 wherein at least about 95% of the Hb in the PolyHb is locked in at least one of the tense or relaxed quaternary state and at least about 95% of the PolyHb has a molecular weight (MW) of at least about 500 kDa.

3. The composition of claim 1 wherein the majority of the hemoglobin (Hb) in the PolyHb is locked in the tense state.

4. The composition of claim 1 wherein the majority of the Hb in the PolyHb is locked in the relaxed state.

5. The composition of claim 1 wherein the PolyHb has a MW in a range from about 500 kDa to about 50,000 kDa.

6. The composition of claim 1 wherein the PolyHb has a cross-linker to Hb molar ratio of at least about 20:1.

7. The composition of claim 6 wherein the cross-linker is selected from the group consisting essentially of glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and combinations thereof.

8. The composition of claim 1 wherein the cross-linker is from at least one of a bisimidate class, the acyl diazide class, or the aryl dihalide class.

9. A process for producing a HBOC composition comprising:
adjusting the pO₂ of a solution containing Hb to a desired level,
polymerizing the Hb with a cross-linker while maintaining the desired pO₂, and
collecting the PolyHb having a MW of at least about 500 kDa wherein a majority of the PolyHb in the HBOC composition has a MW of at least about 500 kDa.

10. The process of claim 9 wherein the desired level is the pO₂ wherein between 0% and about 10% of the Hb is saturated with O₂.

11. The process of claim 9 wherein the desired level is the pO₂ wherein between about 90% and 100% of the Hb is saturated with O₂.

12. The process of claim 9 wherein the pO₂ adjusting step includes any gas liquid exchange technology.

13. The process of claim 12 wherein the pO₂ adjusting step further includes the addition of an oxygen scavenging agent.

14. The process of claim 9 wherein the molar ratio of the cross-linker to Hb is at least about 20:1.

15. The process of claim 9 wherein the Hb is polymerized with a cross-linker selected from the group consisting essentially of glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and combinations thereof.

16. The process of claim 9 wherein the Hb is polymerized with a cross-linker from at least one of a bisimidate class, the acyl diazide class, or the aryl dihalide class.

17. A method of delivering oxygen to a cell comprising exposing the cell to a composition comprising a polymerized hemoglobin (PolyHb) wherein a majority of the hemoglobin (Hb) in the PolyHb is locked in at least one of the tense or relaxed quaternary state and a majority of the PolyHb has a molecular weight (MW) of at least about 500 kDa.

18. The method of claim 17 further comprising including the PolyHb composition with cell culture medium.

19. The method of claim 17 further comprising infusing the PolyHb composition into the cardiovascular system of a subject.

20. The method of claim 17 further comprising perfusing the tissue of a subject with the PolyHb composition.
